(19) United States
(12) Patent Application Publication
(10) Pub. No.: US 2014/0310024 A1
(43) Pub. Date: Oct. 16, 2014

(54) WIFI CLOUD ELECTRONIC VITAL-SIGN MONITORING SYSTEM

(71) Applicant: OSTAR MEDITECH CORP., New Taipei City (TW)

(72) Inventor: Kuo-Yuan CHANG, New Taipei City (TW)

(73) Assignee: OSTAR MEDITECH CORP., New Taipei City (TW)

(21) Appl. No.: 14/031,484

(22) Filed: Sep. 19, 2013

(30) Foreign Application Priority Data
Apr. 12, 2013 (TW) 102112972

(51) Int. Cl. G06F 19/00; A61B 5/00

(52) U.S. Cl. CPC G06F 19/322; A61B 5/68
USPC 705/3

(57) ABSTRACT

A WiFi cloud electronic vital-sign monitoring system has the function of monitoring physiological information and the WiFi transmission unit for transmitting the physiological information or the computation result to a cloud system. Thus, the WiFi cloud electronic vital-sign monitoring system with the WiFi distant transmission function can provide suitable and convenient assistance to the hospitals, the tele-care centers, the nursing homes, the elderly communities and the family care users.

### BACKGROUND OF THE INVENTION

1. Field of the Invention

The present invention relates to a cloud electronic vital-sign monitoring system, and more particularly, to a WiFi cloud electronic vital-sign monitoring system having the WiFi transmission unit to distantly transmit the physiological information or the computation result to a cloud system.

2. Description of the Prior Art

The patients in the hospital, the participators of the tele-care center, the old persons in the nursing home, the residents of the elderly community and the family care users have to monitor their essential physiological information, such as heart information, blood pressure information, oxygen saturation information, blood sugar information, electrocardiogram information, body temperature information, respiration information and body weight information. Generally speaking, the heart information includes heart spectrum information and heart rate information. The physiological information is written out to patients' anamneses by hand in the traditional hospitals, and the electronic anamneses are adopted in the modern hospitals coming with the electronic physiological monitoring system to record the physiological information of patients. However, most physiological information is still observed and written out by the nursing staffs and then inputted into the computer now, and this may cause extra burden of the nursing staffs. Furthermore, the data inaccuracy caused by the busy work or the handing-over of the nursing staffs may make some errors on medicating or treating. Most physiological monitoring instruments on the market or used in the hospitals merely have the function of monitoring, computing and displaying. These instruments can compute and display the result after monitoring the physiological information, but cannot distantly transmit the physiological information or the computation result to a cloud system owing to lacking of the WiFi transmission function.

Therefore, a WiFi cloud electronic vital-sign monitoring system is introduced in the present application that has the WiFi transmission and other additional functions to provide various users with functions of monitoring and managing the physiological information immediately and accurately.

### SUMMARY OF THE INVENTION

It is therefore an objective of the present invention to provide a WiFi cloud electronic vital-sign monitoring system that has the function of monitoring physiological information and further includes the WiFi transmission unit to transmit physiological information or computation result to a cloud system. The WiFi cloud electronic vital-sign monitoring system with distantly WiFi transmitting function can provide suitable and convenient assistance to the hospitals, the tele-care centers, the nursing homes, the elderly communities and the family care users.

For the above-mentioned purpose, according to one embodiment of the present invention, a WiFi cloud electronic vital-sign monitoring system comprises a detecting unit, a computing unit, a displaying unit, a WiFi transmission unit and a power unit. The detecting unit is used for detecting a physiological information of a user. The computing unit is connected to the detecting unit for receiving and computing the physiological information from the detecting unit and providing a computation result. The displaying unit is connected to the computing unit for displaying the physiological information or the computation result. The WiFi transmission unit is connected to the computing unit, and the WiFi transmission unit is wirelessly connected to an external wireless Access Point (AP) to transmit the physiological information or the computation result to a cloud system. The power unit is used for providing a power to the WiFi cloud electronic vital-sign monitoring system.

Furthermore, in one preferred embodiment, the WiFi transmission unit is an internal wireless Access Point having a wireless Access Point mode to let an external electronic device wirelessly connected to the WiFi cloud electronic vital-sign monitoring system through the internal wireless Access Point, and at least an internal parameter of the WiFi cloud electronic vital-sign monitoring system is set or modified directly through the external electronic device.

Furthermore, in one preferred embodiment, the WiFi transmission unit has a WiFi Protected Setup (WPS) mode and the WiFi transmission unit complies with the WiFi Protected Setup (WPS) communication standard.

In one preferred embodiment, the WiFi cloud electronic vital-sign monitoring system further comprises at least one button or one touch panel used for switching the WiFi transmission unit to a wireless Access Point (AP) mode or a WiFi Protected Setup (WPS) mode, or used for turning on or turning off the WiFi transmission unit. Wherein the wireless Access Point mode is used for making the WiFi transmission unit as an internal wireless Access Point, and letting an external electronic device wirelessly connected to the WiFi cloud electronic vital-sign monitoring system through the internal wireless Access Point, and at least an internal parameter of the WiFi cloud electronic vital-sign monitoring system is set or modified directly through the external electronic device. The WiFi transmission unit includes a WiFi Protected Setup (WPS) mode and the mode complies with the WiFi Protected Setup (WPS) communication standard.

In one preferred embodiment, the physiological information is at least one of a heart information, a blood pressure information, an oxygen saturation information, a blood sugar information, an electrocardiogram information, a body temperature information, a respiration information and a body weight information.

In one preferred embodiment, the cloud system is a cloud database, a Hospital Information System (HIS), a Nursing Information System (NIS), an emergency system, an intensive care unit system or an outpatient system.

In one preferred embodiment, the WiFi cloud electronic vital-sign monitoring system further comprises an input unit connected to the computing unit, for a user manually setting connection information of at least an external wireless Access Point through the input unit.

In one preferred embodiment, the WiFi cloud electronic vital-sign monitoring system further comprises a connecting interface connected to the computing unit, for connecting at least one of an external electronic device and a personal profile identifying unit.

In one preferred embodiment, the WiFi cloud electronic vital-sign monitoring system further comprises a determining unit connected to the computing unit, the WiFi transmission unit and the connecting interface. When the determining unit determines the connecting interface is connected to the external electronic device, the WiFi transmission unit will be turned off and the computing unit will transmit the physiological information or the computation result to the external electronic device through the connecting interface.

In one preferred embodiment, the WiFi cloud electronic vital-sign monitoring system further comprises a time synchronizing unit connected to the connecting interface. When the connecting interface is connected to the external electronic device, the time synchronizing unit synchronizes the time with the external electronic device through the connecting interface.

The present invention provides a WiFi cloud electronic vital-sign monitoring system having the function of monitoring physiological information and the WiFi transmission unit for transmitting the physiological information or the computation result to a cloud system. Further, the present invention is capable of providing the wireless Access Point mode, the WiFi Protected Setup mode, and the time synchronizing function according to various requirements. Thus, the present invention can provide suitable and convenient assistance to the hospitals, the tele-care centers, the nursing homes, the elderly communities and the family care users.

The objective, technologies, features and advantages of the present invention will become more apparent from the following description in conjunction with the accompanying drawings, wherein certain embodiments of the present invention are set forth by way of illustration and examples.

### BRIEF DESCRIPTION OF THE DRAWINGS

The foregoing aspects and many of the accompanying advantages of this invention will become more readily appreciated as the same becomes better understood by reference to the following detailed description, when taken in conjunction with the accompanying drawings, wherein:

FIG. 1 is a structurally schematic diagram of the WiFi cloud electronic vital-sign monitoring system according to one embodiment of the present invention.

### DESCRIPTION OF THE PREFERRED EMBODIMENT

The detailed description is provided below and the preferred embodiments described are only for the purpose of description rather than for limiting the present invention.

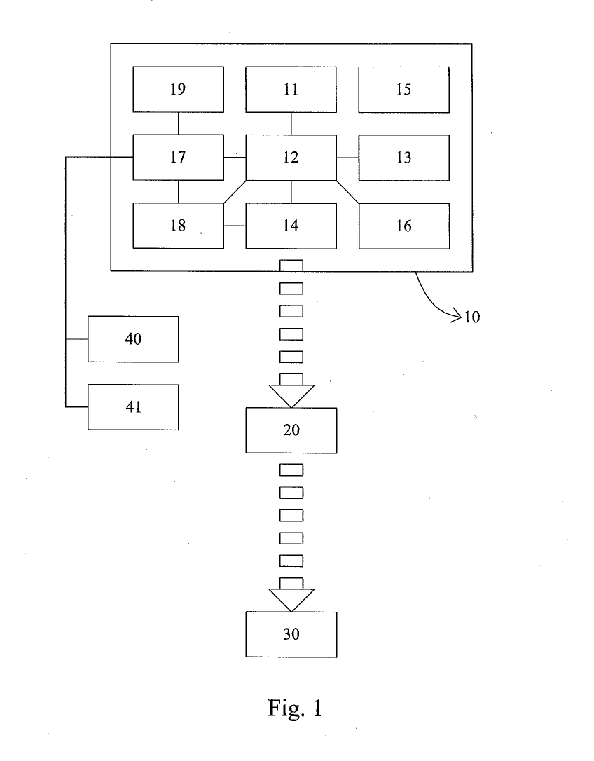

FIG. 1 is a structural diagram of the WiFi cloud electronic vital-sign monitoring system 10 according to one embodiment of the present invention. As shown in FIG. 1, a WiFi cloud electronic vital-sign monitoring system 10 comprises a detecting unit 11, a computing unit 12, a displaying unit 13, a WiFi transmission unit 14 and a power unit 15. The detecting unit 11 is used for detecting a physiological information of a user. The computing unit 12 is connected to the detecting unit 11 for receiving and computing the physiological information from the detecting unit 11 and providing a computation result. The displaying unit 13 is connected to the computing unit 12 for displaying the physiological information or the computation result. The WiFi transmission unit 14 is connected to the computing unit 12, and the WiFi transmission unit 14 is wirelessly connected to an external wireless Access Point (AP) 20 to transmit the physiological information or the computation result to a cloud system 30. The power unit 15 is used for providing a power to the WiFi cloud electronic vital-sign monitoring system 10.

In one preferred embodiment, referring to FIG. 1, the WiFi transmission unit 14 is an internal wireless Access Point having a wireless Access Point mode to make an external electronic device 40 wirelessly connected to the WiFi cloud electronic vital-sign monitoring system 10 through the internal wireless Access Point, and at least an internal parameter of the WiFi cloud electronic vital-sign monitoring system 10 is capable of being set or modified directly through the external electronic device 40. The conventional vital-sign monitoring system does not have the function of the wireless Access Point, and the users have to operate directly on the vital-sign monitoring system to set parameters or other operations, which is not convenient to the users. According to the WiFi cloud electronic vital-sign monitoring system 10 of the present invention, when the wireless Access Point mode is turned on, the users can wirelessly connect the system to modify the parameters or the operation mode, or set at least one wireless Access Point connection information through the external electronic device 40 having the WiFi function, such as a smart phone, a tablet, a laptop or a personal computer. The WiFi cloud electronic vital-sign monitoring system 10 can immediately connect to an external wireless Access Point 20 according to the newly set wireless Access Point connection information, and transmit the physiological information or the computation result to a cloud system 30 through the WiFi function.

In one preferred embodiment, the WiFi transmission unit 14 includes a WiFi Protected Setup (WPS) mode and the WiFi transmission unit 14 complies with the WiFi Protected Setup (WPS) communication standard. When the WiFi Protected Setup mode is turned on, the WiFi cloud electronic vital-sign monitoring system 10 can automatically match and connect to any nearby external wireless Access Point 20 having WiFi Protected Setup (WPS) function through the WiFi transmission unit 14, and the physiological information or the computation result can be transmitted to a cloud system 30 through the WiFi transmission function. Hence, the users can setup the wireless network much easily and safely.

In one preferred embodiment, the WiFi cloud electronic vital-sign monitoring system 10 further comprises at least one button or one touch panel used for switching the WiFi transmission unit 14 to a wireless Access Point (AP) mode or a WiFi Protected Setup (WPS) mode, or for turning on or turning off the WiFi transmission unit 14.

In one preferred embodiment, as shown in FIG. 1, the WiFi cloud electronic vital-sign monitoring system 10 further comprises an input unit 16 connected to the computing unit 12 for a user manually setting at least one wireless Access Point connection information through the input unit 16.

In one preferred embodiment, as shown in FIG. 1, the WiFi cloud electronic vital-sign monitoring system 10 further comprises a connecting interface 17 connected to the computing unit 12, for connecting at least one of an external electronic device 40 and a personal profile identifying unit 41. The connecting interface 17 can be a wired connecting interface (FIG. 1 shows the connection method with a wired connecting interface) or a wireless connecting interface. The wired connecting interface comprises the Universal Serial Bus (USB) terminal or the RS232 terminal, and so on. The wireless connecting interface 17 comprises the Bluetooth, WiFi series wireless communication technology, or Zigbee, and so on. Besides, the external electronic device 40 comprises a smart phone, a tablet, a laptop, or a personal computer. The personal information identifying unit 41 comprises an ID scanner, a barcode scanner, a card reader, a RFID reader, or a Near Field Communication (NFC) device. The connecting interface 17 can be used for identifying the users and the patients, and accurately filing the physiological information or the computation result to avoid error in record.

In one preferred embodiment, as shown in FIG. 1, the WiFi cloud electronic vital-sign monitoring system 10 further comprises a determining unit 18 connected to the computing unit 12, the WiFi transmission unit 14 and the connecting interface 17, wherein when the determining unit 18 determines the connecting interface 17 is connected to an external electronic device 40, the WiFi transmission unit 14 will be turned off and the computing unit 12 will transmit the physiological information or the computation result to the external electronic device 40 through the connecting interface 17.

In one preferred embodiment, as shown in FIG. 1, the WiFi cloud electronic vital-sign monitoring system 10 further comprises a time synchronizing unit 19 connected to the connecting interface 17, wherein when the connecting interface 17 is connected to an external electronic device 40, the time synchronizing unit 19 will synchronize the time with the external electronic device 40 through the connecting interface 17. The conventional vital-sign monitoring system doesn't have the function of time synchronizing, so that the time error and inaccuracy will happen after a period of time. Using the blood pressure detection as example, generally speaking, the conventional vital-sign monitoring systems used in hospitals, clinics and tele-care centers will have time error after using a period of time or setting fault, so that the measuring time will not be identical in the same organization. For example, the patient has to take medicines on schedule according to the detection result of blood pressure, and the doctor will prescribe according to the detection result of blood pressure. If a time gap exists between the blood pressure detection system and the doctor's prescription, the doctor will possibly make wrong judgment or give wrong prescription, the patient will possibly take wrong medicine, the record of doctor's prescription will possibly earlier than the patient's detection time, the nursing staffs will possibly be confused, and the patients will possibly take hypertension medicine when his blood pressure is low. These situations will cause an awful care quality of the hospitals, and may further make the patients in danger. The WiFi cloud electronic vital-sign monitoring system 10 of the present invention provides the function of time synchronizing that can prevent medical errors or mistakes caused by the time gap, improve the care quality of hospitals, and maintain patient's interests.

In one preferred embodiment, the physiological information is at least one of a heart information, a blood pressure information, an oxygen saturation information, a blood sugar information, an electrocardiogram information, a body temperature information, a respiration information and a body weight information. The heart information includes heart spectrum information and heart rate information. Using the heart spectrum information for example, the heart spectrum information is detecting the heart information of a human and transferring the heart information into a spectrum. According to U.S. Pat. No. 7,135,000, the fast Fourier transform (FFT) algorithm is used to transfer the spectrum, and the spectrum generally has 3 to 5 main frequency waveforms. The apex of the first main frequency waveform is the heartbeat frequency of a human. If some noisy waveforms appear beside the main frequency waveform, the heartbeat of the patient is an irregular situation and will be treated as abnormal. So that the heart spectrum information can be used for determining the situation of a patient's heart.

In one preferred embodiment, the cloud system 30 is capable of being a cloud database, a Hospital Information System (HIS), a Nursing Information System (NIS), an emergency system, an intensive care unit system or an outpatient system. The cloud system 30 also complies with the standard of Health Level 7 (HL7) that can exchange or transfer information between different cloud system 30.

To sum up, the present invention provides a WiFi cloud electronic vital-sign monitoring system having the function of detecting physiological information and the WiFi transmission unit for transmitting the physiological information or the computation result to a cloud system. Furthermore, the system provides wireless Access Point mode, WiFi protected Setup mode, and time synchronizing mode to match different requirements. Hence, the system can provide suitable and convenient assistance to the hospitals, the tele-care centers, the nursing homes, the elderly communities and the family care users.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the spirit and scope of the appended claims.

What is claimed is:

1. A WiFi cloud electronic vital-sign monitoring system comprising:
a detecting unit, for detecting a physiological information of a user;
a computing unit, connected to the detecting unit for receiving and computing the physiological information from the detecting unit and providing a computation result;
a displaying unit, connected to the computing unit for displaying the physiological information or the computation result;
a WiFi transmission unit, connected to the computing unit, the WiFi transmission unit is wirelessly connected to an external wireless Access Point to transmit the physiological information or the computation result to a cloud system; and
a power unit, for providing a power to the WiFi cloud electronic vital-sign monitoring system.

2. The WiFi cloud electronic vital-sign monitoring system of claim 1, wherein the WiFi transmission unit is an internal wireless Access Point having a wireless Access Point mode to let an external electronic device wirelessly connected to the WiFi cloud electronic vital-sign monitoring system through the internal wireless Access Point, and at least an internal parameter of the WiFi cloud electronic vital-sign monitoring system is set or modified directly through the external electronic device.

3. The WiFi cloud electronic vital-sign monitoring system of claim 1, wherein the WiFi transmission unit has a WiFi Protected Setup mode and the WiFi Protected Setup mode complies with the WiFi Protected Setup communication standard.

4. The WiFi cloud electronic vital-sign monitoring system of claim 1 further comprising at least a button or a touch panel used for switching the WiFi transmission unit to a wireless Access Point mode or a WiFi Protected Setup mode, or used for turning on or turning off the WiFi transmission unit, wherein the wireless Access Point mode of the WiFi transmission unit is used for making the WiFi transmission unit as an internal wireless Access Point, and letting an external electronic device wirelessly connected to the WiFi cloud electronic vital-sign monitoring system through the internal wireless Access Point, and at least an internal parameter of the WiFi cloud electronic vital-sign monitoring system is set or modified directly through the external electronic device; and the WiFi transmission unit has a WiFi Protected Setup mode and the WiFi Protected Setup mode complies with the WiFi Protected Setup communication standard.

5. The WiFi cloud electronic vital-sign monitoring system of claim 1, wherein the physiological information is at least one of a heart information, a blood pressure information, an oxygen saturation information, a blood sugar information, an electrocardiogram information, a body temperature information, a respiration information and a body weight information.

6. The WiFi cloud electronic vital-sign monitoring system of claim 1, wherein the cloud system is a cloud database, a Hospital Information System, a Nursing Information System, an emergency system, an intensive care unit system or an outpatient system.

7. The WiFi cloud electronic vital-sign monitoring system of claim 1 further comprising an input unit connected to the computing unit, for a user manually setting connection information of at least an external wireless Access Point through the input unit.

8. The WiFi cloud electronic vital-sign monitoring system of claim 1 further comprising a connecting interface connected to the computing unit, for connecting at least one of an external electronic device and a personal profile identifying unit.

9. The WiFi cloud electronic vital-sign monitoring system of claim 8 further comprising a determining unit connected to the computing unit, the WiFi transmission unit and the connecting interface, wherein when the determining unit determines the connecting interface is connected to the external electronic device, the WiFi transmission unit is turned off and the computing unit will transmit the physiological information or the computation result to the external electronic device through the connecting interface.

10. The WiFi cloud electronic vital-sign monitoring system of claim 8 further comprising a time synchronizing unit connected to the connecting interface, wherein when the connecting interface is connected to the external electronic device, the time synchronizing unit synchronizes the time with the external electronic device through the connecting interface.
